(19) United States
(12) Patent Application Publication
(10) Pub. No.: US 2025/0302708 A1
(43) Pub. Date: Oct. 2, 2025

(54) METHOD AND COMPOSITION FOR BIOFILM REMOVAL

(71) Applicant: ProEdge Dental Products, Inc., Centennial, CO (US)

(72) Inventors: William CARR, Port St. Lucie, FL (US); Mark FRAMPTON, Centennial, CO (US)

(21) Appl. No.: 19/092,282

(22) Filed: Mar. 27, 2025

Related U.S. Application Data
(60) Provisional application No. 63/570,520, filed on Mar. 27, 2024.

(51) Int. Cl. A61K 8/27; A61K 8/365; A61K 8/41; A61K 8/44; A61K 8/86; A61Q 11/00

(52) U.S. Cl. CPC A61K 8/27; A61K 8/365; A61K 8/416; A61K 8/44; A61K 8/86; A61Q 11/00; A61K 2800/87

(57) ABSTRACT

A biofilm shock treatment includes: a zinc ion source and a disinfecting agent. The biofilm shock treatment is configured to weaken a matrix of a biofilm and to disinfect causative microbes while promoting biofilm removal. Optionally, the biofilm shock may include a surfactant and/or a chelating agent.

### CROSS REFERENCE TO RELATED APPLICATIONS

This application claims the benefit of priority pursuant to 35 U.S.C. § 119(e) of U.S. Provisional Application No. 63/570,520 filed on Mar. 27, 2024, titled “METHOD AND COMPOSITION FOR BIOFILM REMOVAL” which is hereby incorporated by reference herein in its entirety.

### BACKGROUND

Biofilms have increasingly become a significant concern in various industries, including dentistry, healthcare, potable water treatment, food service and processing, agriculture, manufacturing, and other industrial processes. In one example in dental water lines, the formation of biofilms poses substantial challenges and potential risks. Biofilms in dental water lines can lead to the proliferation of microorganisms, including pathogenic non-tuberculosis mycobacteria (NTMs). These persistent colonies of bacteria adhere to the inner surfaces of water lines, creating a complex and resilient ecosystem that can compromise the quality of dental water and contribute to the development of infections. Outbreaks of NTMs in dental water lines have been reported, highlighting the adverse consequences of biofilm formation. NTMs are opportunistic pathogens capable of causing severe infections, particularly in immunocompromised individuals. The presence of biofilms and NTMs in dental water lines poses a serious threat to patient safety and emphasizes the critical need for effective solutions to prevent biofilm formation and mitigate the associated risks. As such, addressing the problems caused by biofilms in dental water lines, particularly in the context of NTM outbreaks, is essential for ensuring the safety and efficacy of dental procedures. Developing innovative technologies and methodologies to control biofilm formation and eliminate NTMs in dental water lines is crucial to safeguarding public health and promoting best practices in dental care.

The resistance of biofilms to traditional cleaning agents may be attributed to the hydrophobic nature of the extracellular polymeric substance (EPS) layer of the biofilm matrix, which encapsulates and protects the microbial community within the biofilm matrix. This EPS layer creates a barrier that hinders the penetration of disinfectants and cleaning agents, making it challenging to effectively eradicate biofilms using conventional approaches. The hydrophobic properties of the EPS make it difficult for aqueous solutions to permeate the biofilm structure, limiting the contact between the cleaning agents and the embedded microorganisms. Consequently, biofilms in dental water lines exhibit elevated resilience to standard disinfection methods, contributing to the persistence of microbial contamination and the potential for NTM outbreaks. Even if disinfectants are able to kill the bacteria, often a portion of the EPS layer and dead cells are left adhering to the walls of the waterline, which enable rapid regrowth of the biofilm. Improved compounds and methods to kill and remove biofilms from dental water lines and other equipment is needed.

### BRIEF SUMMARY

In one embodiment, a biofilm shock treatment, includes: a zinc ion source, a disinfecting agent, where the biofilm shock treatment is configured to weaken a matrix of a biofilm and to disinfect causative microbes while promoting biofilm removal.

Optionally, in some embodiments, the biofilm shock treatment may also include a chelating agent.

Optionally, in some embodiments, the biofilm shock treatment may also include a surfactant.

Optionally, in some embodiments, the zinc ion source is configured to weaken the matrix of the biofilm.

Optionally, in some embodiments, the zinc ion source includes at least one of zinc acetate dihydrate, zinc lactate, or zinc oxide.

Optionally, in some embodiments, the biofilm shock treatment further comprises an organic acid.

Optionally, in some embodiments, the organic acid comprises lactic acid.

Optionally, in some embodiments, the zinc ion source dissociates into a zinc ion and an organic acid.

Optionally, in some embodiments, the zinc ion source is between about 1.4 to 5 mass percent of the biofilm shock treatment.

Optionally, in some embodiments, the zinc ion source reduces a hydrophobicity property of the biofilm, enhancing the access of the biofilm shock treatment to the biofilm.

Optionally, in some embodiments, the disinfecting agent includes one or more ammonium chlorides.

In one embodiment, a method of removing a biofilm, includes: applying a biofilm shock solution to a surface having a biofilm, the biofilm shock solution including a zinc ion source, and a disinfecting agent.

Optionally, in some embodiments, the biofilm shock further includes a chelating agent.

Optionally, in some embodiments, the biofilm shock further includes a surfactant.

Optionally, in some embodiments, the surface includes a conduit in a dental waterline system.

Optionally, in some embodiments, the method of removing a biofilm includes allowing the biofilm shock solution to interact with the biofilm for a predetermined period to weaken a matrix of the biofilm; and removing the weakened biofilm from the surface.

In one embodiment, a method of making a biofilm shock solution, includes: combining a zinc ion source, a disinfecting agent, and water to form the biofilm shock solution.

Optionally, in some embodiments, the method of making the biofilm shock solution includes combining a chelating agent into the biofilm shock.

Optionally, in some embodiments, the method of making the biofilm shock solution includes combining a surfactant into the biofilm shock.

Optionally, in some embodiments, the method of making the biofilm shock solution includes selecting the disinfecting agent based on a type of targeted microbe.

Optionally, in some embodiments, the chelating agent is between about 1.3 and 14.7 mass percent of the biofilm shock treatment.

Optionally, in some embodiments, the disinfecting agent is between about 0.15 and 2.25 mass percent of the biofilm shock treatment.

Optionally, in some embodiments, the zinc ion source reduces the hydrophobicity of an extracellular polymeric substance of the biofilm.

Optionally, in some embodiments, the chelating agent includes an EDTA compound.

Optionally, in some embodiments, the EDTA chelates a mineral deposit within a matrix of the biofilm.

Optionally, in some embodiments, the ammonium chloride includes a quaternary ammonium solution.

Optionally, in some embodiments, the surfactant is a non-ionic surfactant.

Optionally, in some embodiments, the surfactant includes at least one of polyethylene glycol, octoxynol-9, octoxynol-10, or octoxynol-12, alkyl glucosides, alcohol ethoxylates, block polymers, or a pluronic nonionic surfactant.

Optionally, in some embodiments, the zinc ion source is between about 1.4 to 5 mass percent of the biofilm shock solution, the chelating agent is between about 1.3 and 14.7 mass percent of the biofilm shock solution, and the disinfecting agent is between about 0.15 and 2.25 mass percent of the biofilm shock solution.

Optionally, in some embodiments, the zinc ion source is between about 1.4 to 5 mass percent of the biofilm shock solution, the chelating agent is between about 1.3 and 14.7 mass percent of the biofilm shock solution, and the disinfecting agent is between about 0.15 and 2.25 mass percent of the biofilm shock solution.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 is a schematic of a use case for a biofilm shock.

FIG. 2A is an example of a conduit which may be subject to biofilm growth.

FIG. 2B is a detail view of a wall of the conduit of FIG. 2A showing an example of a biofilm.

FIG. 3A is a further detail view of a wall of the conduit of FIG. 2A showing the biofilm of FIG. 2B including microbes and a biofilm matrix including an extracellular polymeric substance layer.

FIG. 3B illustrates treatment of the biofilm of FIG. 3AA with a biofilm shock and weakening of the matrix of the biofilm.

FIG. 3C illustrates the conduit of FIG. 3AA after treatment with the biofilm shock and removal of the extracellular polymeric substance layer.

FIG. 4 illustrates test results of the use of embodiments of a biofilm shock to eliminate a representative biofilm.

FIG. 5A is a micrograph of a surface with a biofilm before treatment with a biofilm shock.

FIG. 5B is a micrograph of the surface of FIG. 5A after treatment with a biofilm shock.

FIG. 6A is a micrograph of a surface with a biofilm before treatment with a biofilm shock.

FIG. 6B is a micrograph of the surface of FIG. 6A after treatment with a biofilm shock.

FIG. 7A is a micrograph of a surface with a biofilm before treatment with a biofilm shock.

FIG. 7B is a micrograph of the surface of FIG. 7A after treatment with a biofilm shock.

FIG. 8A is a micrograph of a surface with a biofilm before treatment with an existing commercial product.

FIG. 8B is a micrograph of the surface of FIG. 8A after treatment with the existing commercial product.

FIG. 9 is a flow chart of a method of removing a biofilm.

### DETAILED DESCRIPTION

Novel methods and compositions for removal of biofilms are disclosed. In particular, biofilm shock solutions are disclosed that can disrupt the structure of the biofilm such as an EPS layer and/or mineral deposits, disinfect the causative microbes, and flush the weakened biofilm from the equipment. The biofilm shocks are generally prepared as an aqueous solution that can be sprayed, pumped, or otherwise applied to a surface from which a biofilm is to be removed. The biofilm shock may be allowed to soak a surface for a certain time (e.g., about 10 minutes, several hours, or overnight) before being flushed away. For example, the biofilm shock may be left in contact with the surface for several minutes, several hours, overnight, or for a day or more.

As mentioned, the EPS layer of a biofilm protects the bacteria or other microorganisms that form or inhabit a biofilm from disinfecting agents. One mechanism of that protection is hydrophobicity. The EPS layer is typically hydrophobic such that it repels water and aqueous solutions such that the solutions cannot wet the EPS layer and attack the underlying microbes. The hydrophobicity of an EPS layer is sometimes referred to as being rose-like or lotus-like superhydrophobicity (named for the extremely strong hydrophobicity observed in the respective plants). EPS layers may also include structural components such as fibers formed by fiber-forming proteins and deposited minerals that also contribute to the structural stability of the biofilm.

In various embodiments, the biofilm shock includes a zinc ion source such as zinc acetate dihydrate. Other zinc compounds that produce zinc ions in solution may be used in addition to, or instead of, zinc acetate dihydrate, such as zinc lactate, zinc oxide, etc. The zinc ion source may act to reduce or break down the hydrophobicity of the EPS layer. Other metal ion sources, such as copper, silver, or titanium may reduce hydrophobicity as well.

In embodiments where the zinc ion source comprises zinc lactate and/or zinc lactate dihydrate, the zinc lactate dissociates in an aqueous solution according to the following reaction:

Zn(C₃H₅O₃)₂(s)→Zn²⁺(aq)+2[C₃H₅O₃]⁻(aq)

Thus, some embodiments of the biofilm shock may include an organic acid (e.g., lactic acid) or another acid. In some embodiments, the acid may be formed by the dissociation of the zinc ion source in an aqueous solution. For example, where the zinc ion source comprises zinc lactate, the shock includes both free zinc ions and lactic acid. Either or both of the zinc ions or the lactic acid may help disable, clean, and/or remove the biofilm. For example, the lactic acid may penetrate the EPS layer of the biofilm, thereby destabilizing the film and enabling the disinfecting agent to access the microbes forming the film. In other examples, lactic acid may lower the pH of the aqueous solution which can inhibit the growth and survival of the biofilm-forming microbes. Lactic acid may alter surface properties of the biofilm-forming microbes and interfere with their ability to accumulate and agglomerate on surfaces, thereby preventing the formation of biofilms. Some biofilms are formed by microbes that exhibit quorum sensing mechanisms by which microbes coordinate activity. Lactic acid may interfere with quorum sensing mechanisms, thereby weakening biofilms or preventing their formation.

In various embodiments, the biofilm shock includes a disinfecting agent 316 or microbe-killing agent. In one example, the disinfecting agent may be an ammonium chloride or mixture of ammonium chlorides, possibly with other chemicals, such as, but not limited to, Alkyl Dimethyl Benzyl Ammonium Chloride, Di-n-Octyl Dimethyl Ammonium Chloride, n-Octyl-Decyl Dimethyl Ammonium Chloride, and/or Di-n-Decyl Dimethyl Ammonium Chloride. In some embodiments, the disinfecting agent may be a quaternary ammonium solution. In some embodiments, a different disinfecting agent may be used as a replacement for, or in combination with, an ammonium chloride, which may be selected based on a type of microbe being targeted, the setting (e.g., industrial, medical, dental, etc.).

In various embodiments, the biofilm shock optionally includes a chelating agent such as ethylene diamine tetra acetate (“EDTA”). Chelating agents are chemical compounds that have the ability to form stable complexes with metal ions by binding to them through multiple coordination sites. Their ability to form stable complexes with metal ions allows chelating agents to disrupt mineral deposits within the biofilm, thereby weakening the structure of the biofilm. For example, the microbes that create biofilms may deposit calcium or magnesium oxides such as calcium carbonate or magnesium oxide to aid in stability of the biofilm. The chelating agent may disrupt the calcium oxide by binding to the calcium, thereby wreaking the physical structure of the biofilm. The chelating agent may also act as a sequestrant, preventing the minerals in the mineral deposit from re-depositing elsewhere.

Where EDTA is used as the chelating agent 312, it may be used in the form of disodium EDTA (e.g., Na₂EDTA or CaNa₂EDTA), tetrasodium EDTA (Na₄EDTA), or EDTA acid (ethylene diamine tetra acetic acid), the former two being preferred to help keep the pH of the biofilm shock 306 in the desired range of neutral to slightly alkaline (e.g., about 7 to 12).

Other chelating agent 312 that may be used in addition to, or in place of, EDTA include but are not limited to etidronic acid, trisodium ethylenediamine disuccinate, nitrilotriacetic acid, sodium phytate, tetrasodium glutamate diacetate, and/or sodium gluconate.

In various embodiments, the biofilm shock 306 may optionally include a surfactant. A surfactant may aid in wetting the biofilm to enhance the ability of the other components of the biofilm shock to access the biofilm and perform their desired functions. For example, the biofilm shock may include a surfactant such as polyethylene glycol. In embodiments where an ammonium-based disinfecting agent is used, a non-ionic surfactant is preferred because an ionic surfactant, particularly an anionic surfactant, could react with the cations of the ammonium and the resulting compound precipitate out of solution. Other surfactants 314 include, but are not limited to octoxynol-9, octoxynol-10, or octoxynol-12, alkyl glucosides, alcohol ethoxylates, block polymers, and/or pluronic nonionic surfactants.

In various embodiments, the biofilm shock may optionally include one or more of a fragrance and/or a color. While such constituents may not have an effect on the biofilm itself, they can contribute to a user satisfaction with the biofilm shock, such as by having a fresh, clean scent and a pleasing color. In some examples, a mint or evergreen fragrance may be used.

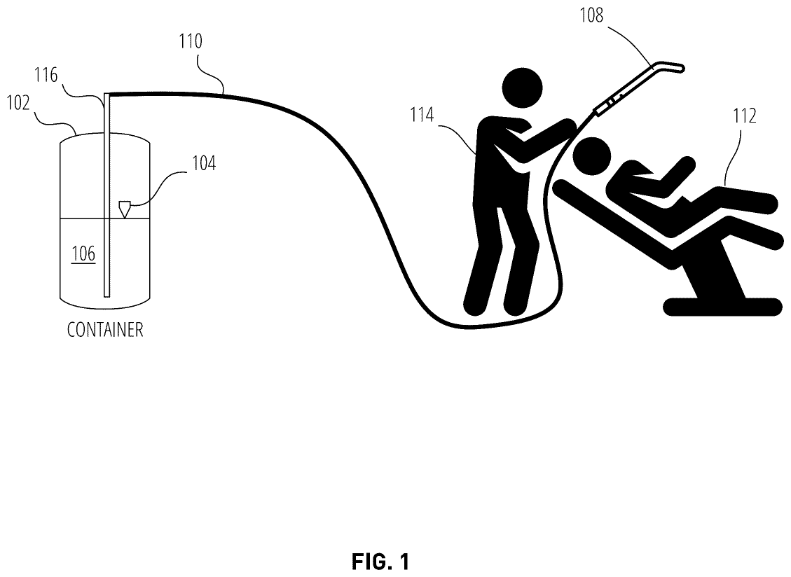

Turning to the figures, FIG. 1 illustrates one example of a use case for the biofilm shock, namely in the context of a dental procedure. Dentists 114 often use a fluid 106, such as water, to irrigate a patient's 112 mouth when performing dental procedures such as filling cavities, extracting teeth, debriding gum tissue, orthodontia, or even in routine cleanings. Typically, city potable water is not used, but the fluid 106 is supplied from a container 102. Often, air pressure is applied to the top of the container 102, which causes a piston effect on a surface 104 of the fluid 106, forcing the fluid out of a tube 116 into a conduit 110. From there, the fluid 106 is delivered to a syringe 108 (often a combination air/water syringe) to be used to irrigate the patient 112. The container 102, tube 116, conduit 110, and the syringe 108 are all susceptible to contamination from a biofilm 202.

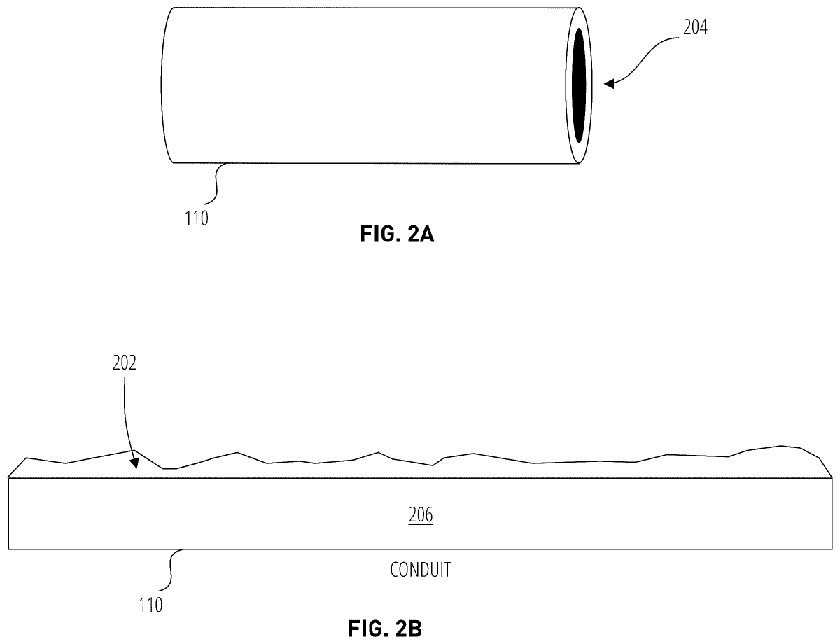

As shown in FIG. 2A and FIG. 2B, an example of a section of the conduit 110 or the tube 116, may develop a biofilm 202. The biofilm 202 may have a rough surface that can harbor pathogens and also narrows the lumen 204 of the conduit 110, decreasing its flowrate. A biofilm 202 may grow thicker over time, exacerbating these effects.

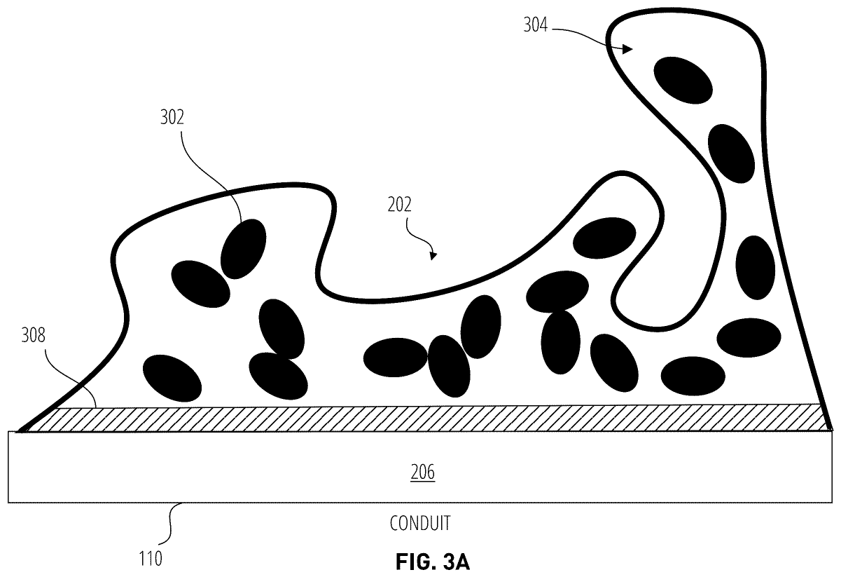

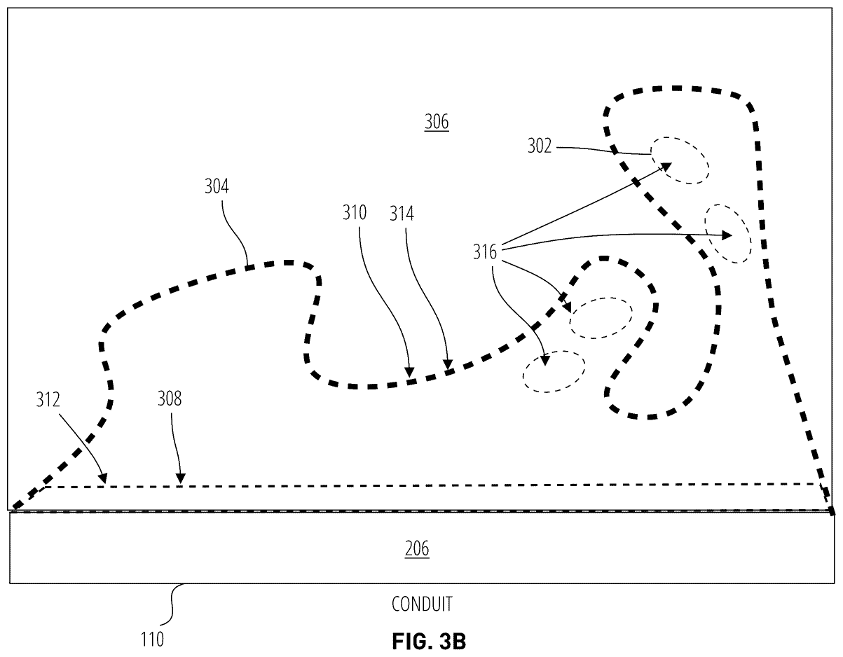

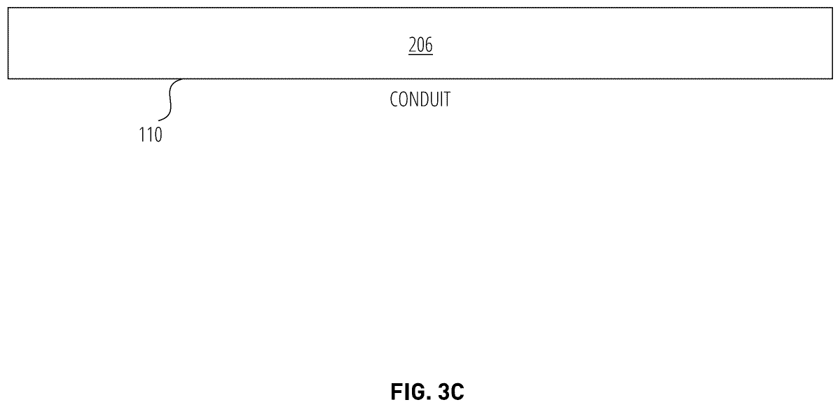

Turning to FIG. 3A, a microscopic view of the example biofilm 202 is shown. The biofilm 202 may be formed by one or more types of microbes 302, or may be formed by some microbes 302, while others are merely present and/or flourish in the biofilm 202. While bacteria are common in forming biofilms, other microbes such as viruses, fungi, archaea, protists (e.g., algae), etc. may be present. The biofilm 202 may have an extracellular polymeric substance 304, as discussed. The microbes 302 may generate a mineral deposit 308 that may be on the wall 206 of the conduit 110 or may be distributed in the biofilm 202. The extracellular polymeric substance 304 and the mineral deposit 308 may protect the biofilm 202 from cleaning and disinfecting agents, as discussed due to the hydrophobicity of the extracellular polymeric substance 304 and/or the structural strength provided by the mineral deposit 308.

FIG. 3B shows an effect of a biofilm shock 306 of the present disclosure on the biofilm 202. The zinc ion source 310 and the surfactant 314 attack the hydrophobicity of the extracellular polymeric substance 304. The chelating agent 312 attacks the mineral deposit 308. These actions weaken the biofilm 202 and enable the disinfecting agent 316 to kill the microbes 302. These weakening and killing actions are indicated by the dashed lines in FIG. 3B. The biofilm shock 306 and/or another fluid such as water may be flushed through the conduit 110 during or after the biofilm shock 306 treatment to wash away the weakened and killed biofilm 202. The conduit 110 following the biofilm shock 306 treatment may be substantially free from the biofilm 202.

Various embodiments of the biofilm shock 306 may include a zinc ion source 310, a chelating agent 312, a surfactant 314, and a disinfecting agent 316, and water in an aqueous solution. The biofilm shock 306 may also include small amounts of other compounds such as colors or fragrances. Representative relative amounts of the components of the biofilm shock 306 are shown in Table 1.

TABLE 1
Ranges of components of embodiments
of the biofilm shock 306

| | mass percent | mass percent |
|---|---|---|
| | (approximate | (approximate |
| | lower | upper |
| Component | range) | range) |
| zinc ion source 310 | 1.4 | 5 |
| chelating agent 312 (optional) | 1.3 | 14.7 |
| surfactant 314 (optional) | 0.5 | 5.0 |
| disinfecting agent 316 | 0.15 | 2.25 |
| water | 77 | 86 |

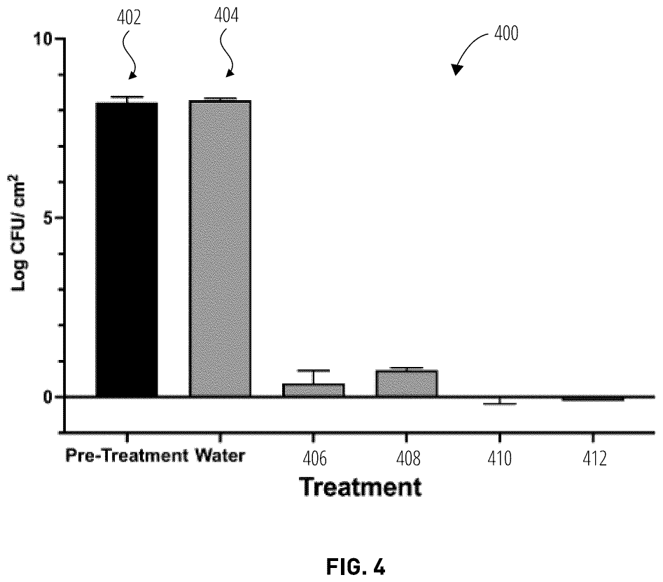

FIG. 4 shows test results 400 using three embodiments (embodiment 406, embodiment 408, and embodiment 410) of the biofilm shock 306, an existing commercial product 412, and a water 404 control treatment against a Pseudomonas aeruginosa biofilm 202. The vertical axis shows log₁₀ colony forming units (CFU) per cm² of a sample dental water conduit 110. CFU/cm² values are shown for pre-treatment condition 402, water 404 control, and for the embodiments 406, 408, and 410 of the biofilm shock 306, as well as the existing commercial product 412. These data are also represented in Table 2.

TABLE 2
Summary statistics of viable cell counts for test results 400.

| | Mean | | Mean |
|---|---|---|---|
| | Log₁₀ | Std. | Log |
| Sample | (CFU/cm²) | Dev. | Reduction |
| Pre-treatment control | 8.222 | 0.155 | N/A |
| Post-treatment water control | 8.285 | 0.052 | −0.063 |
| Embodiment 406 | 0.385 | 0.348 | 7.837 |
| Embodiment 408 | 0.745 | 0.072 | 7.477 |
| Embodiment 410 | −0.017 | 0.174 | 8.239 |
| existing commercial product 412 | −0.117 | 0.000 | 8.339 |

As shown in FIG. 4 and Table 1, there is a significant reduction in biofilm 202 activity as measured by CFU/cm² from use of the biofilm shock 306.

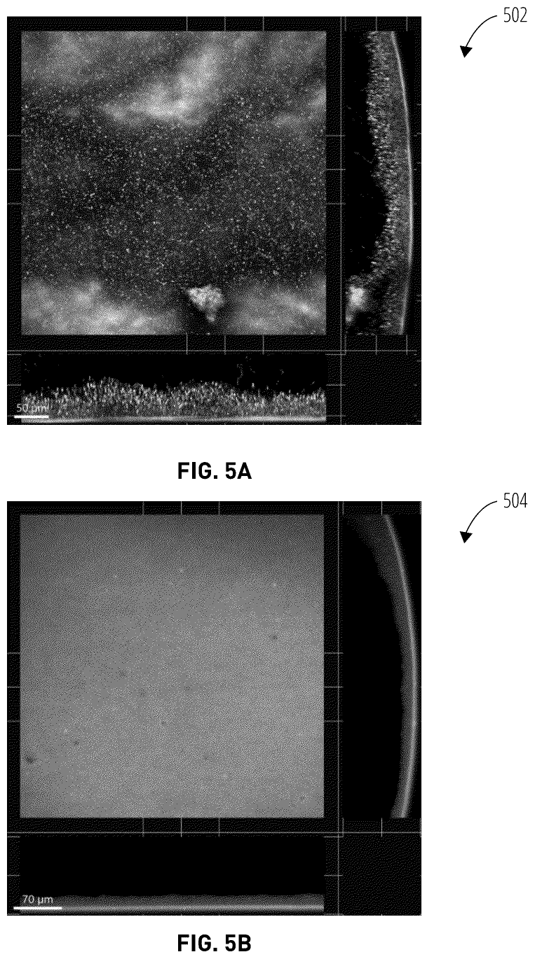

FIG. 5A-FIG. 8B are micrographs (25× magnification) of test samples whose data are represented in the test results 400 shown in FIG. 4 and Table 2.

FIG. 5A shows a pre-treatment condition 502, and FIG. 5B shows a post-treatment condition 504 of a conduit 110 on which a biofilm 202 has been grown. FIG. 5A and FIG. 5B show results from treatment with embodiment 406 of the biofilm shock 306 of a conduit 110 on which a biofilm 202 was grown. The embodiment 406 includes a quaternary ammonium solution as the disinfecting agent 316, EDTA as a chelating agent 312, and polyethylene glycol as the surfactant 314. The embodiment 406 did not include a zinc ion source. FIG. 5A is a plan view (central), edge (lower and right inset) of a conduit 110 surface at 25× magnification, showing a biofilm 202 before treatment with embodiment 406 of the biofilm shock 306. Green areas are viable CFUs and red areas may be inactive or dead areas of the biofilm 202, such as the extracellular polymeric substance 304. FIG. 5B is the same surface as FIG. 5A after treatment with the embodiment 406 of the biofilm shock 306.

As shown for example in FIG. 5A and FIG. 5B, a significant amount of biofilm remained on the surfaces following treatment. Although there is a large reduction in the number of viable cells in the biofilm (7.837 mean log reduction), the micrographs indicate that much of the dead biofilm 202 was still attached. In this case, the biofilm 202 was killed by the embodiment 406 but was largely not removed. This lack of removal has implications for biofilm 202 regrowth; dead biofilm 202 remaining on a surface will promote more rapid biofilm 202 regrowth than a clean surface.

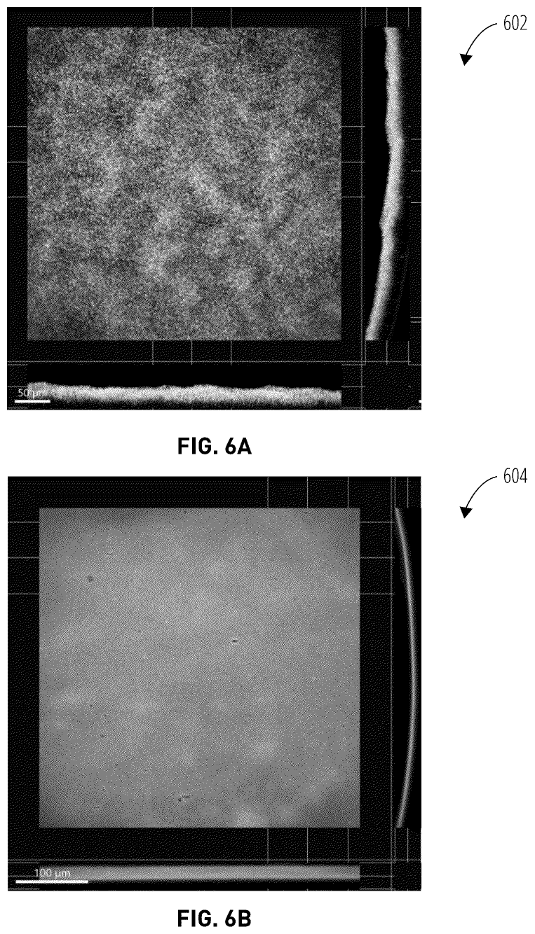

FIG. 6A shows a pre-treatment condition 602, and FIG. 6B shows a post-treatment condition 604 of a conduit 110 on which a biofilm 202 has been grown. FIG. 6A and FIG. 6B show results from treatment with embodiment 408 of the biofilm shock 306 of a conduit 110 on which a biofilm 202 was grown. The embodiment 408 includes a quaternary ammonium solution as the disinfecting agent 316, EDTA as a chelating agent 312, polyethylene glycol as the surfactant 314, and zinc oxide as the zinc ion source 310. The embodiment 406 did not include a zinc ion source. FIG. 6AA is a plan view (central), edge (lower and right inset) of a conduit 110 surface at 25× magnification, showing a biofilm 202 before treatment with embodiment 406 of the biofilm shock 306. Green areas are viable CFUs and red areas may be inactive or dead areas of the biofilm 202, such as the extracellular polymeric substance 304. FIG. 6B is the same surface as FIG. 6A after treatment with the embodiment 408 of the biofilm shock 306.

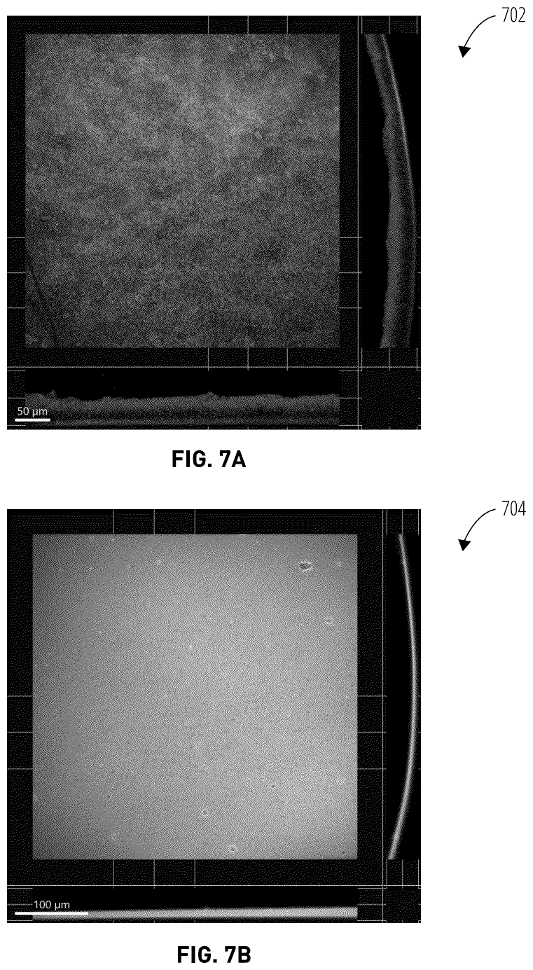

In contrast to FIG. 5A and FIG. 5B, FIG. 6A and FIG. 6B show the surprising result that there is minimal visible biofilm contamination after treatment. The texture observed in FIG. 6B is consistent with the extruded surface of a new conduit 110. These data illustrate the difference between biofilm killing (e.g., FIG. 5A and FIG. 5B) and biofilm removal (e.g., FIG. 6A and FIG. 6B). In the case of the embodiment 408, there was a killing component of the biofilm 202, however there was also removal of the biofilm 202 from the conduit 110. This removal is important for preventing or reducing the re-growth of biofilms 202 which can more-easily re-inhabit dead biofilm 202 structures than a clean surface. these figures show the surprising result that the biofilm 202 is nearly completely eradicated in FIG. 7B. See also the log mean reduction in CFUs of 7.447 in Table 2. Thus, the biofilm shocks disclosed herein show surprising effectiveness against biofilms.

FIG. 7A shows a pre-treatment condition 702, and FIG. 7B shows a post-treatment condition 704 of a conduit 110 on which a biofilm 202 has been grown. FIG. 7A-FIG. 7B show results from treatment with embodiment 410 of the biofilm shock 306 of a conduit 110 on which a biofilm 202 was grown. The embodiment 410 includes a quaternary ammonium solution as the disinfecting agent 316, EDTA as a chelating agent 312, polyethylene glycol as the surfactant 314, and zinc lactate as the zinc ion source 310 (as compared to zinc oxide in the embodiment 408). FIG. 7A is a plan view (central), edge (lower and right inset) of a conduit 110 surface at 25× magnification, showing a biofilm 202 before treatment with embodiment 410 of the biofilm shock 306. FIG. 7B is the same surface as FIG. 7A after treatment with the embodiment 410 of the biofilm shock 306.

As with the embodiment 408, there is both killing and removal of the biofilm 202 with the embodiment 410. In contrast to FIG. 5A and FIG. 5B, FIG. 7A and FIG. 7B show the surprising result that there is minimal visible biofilm contamination after treatment. The texture observed in FIG. 7B is consistent with the extruded surface of a new conduit 110. These data illustrate the difference between biofilm killing (e.g., FIG. 5A and FIG. 5B) and biofilm removal (e.g., FIG. 7A and FIG. 7B). In the case of the embodiment 410, there was a killing component of the biofilm 202, however there was also removal of the biofilm 202 from the conduit 110. This removal is important for preventing or reducing the re-growth of biofilms 202 which can more-easily re-inhabit dead biofilm 202 structures than a clean surface. These figures show the surprising result that the biofilm 202 is nearly completely eradicated in FIG. 7B. See also the log mean reduction of 8.239 in Table 2. Thus, the biofilm shocks disclosed herein show surprising effectiveness against biofilms.

The biofilm shocks herein advantageously remove biofilms in a wide variety of industries where biofilms are a problem, including, but not limited to, dentistry, healthcare, potable water treatment, food service and processing, agriculture, manufacturing, and other industrial processes. The disclosed compositions effectively penetrate the biofilm matrix, overcoming the impermeability barrier posed by the EPS layer and promote thorough removal of biofilms, reducing the risk of recontamination. The biofilm shock can be applied (e.g., by spraying, flushing, soaking, etc.) to various surfaces and settings where biofilms are problematic, including medical facilities, food processing plants, and water distribution systems.

The disclosed methods and compositions provide an effective solution for biofilm removal by addressing the challenges posed by the EPS layer, deposited minerals, and other defenses used by biofilms. The synergistic action of the zinc ion source 310, chelating agent 312, surfactant 314, and disinfecting agent 316 ensures thorough removal of biofilms, making it a valuable tool in various industries requiring biofilm control.

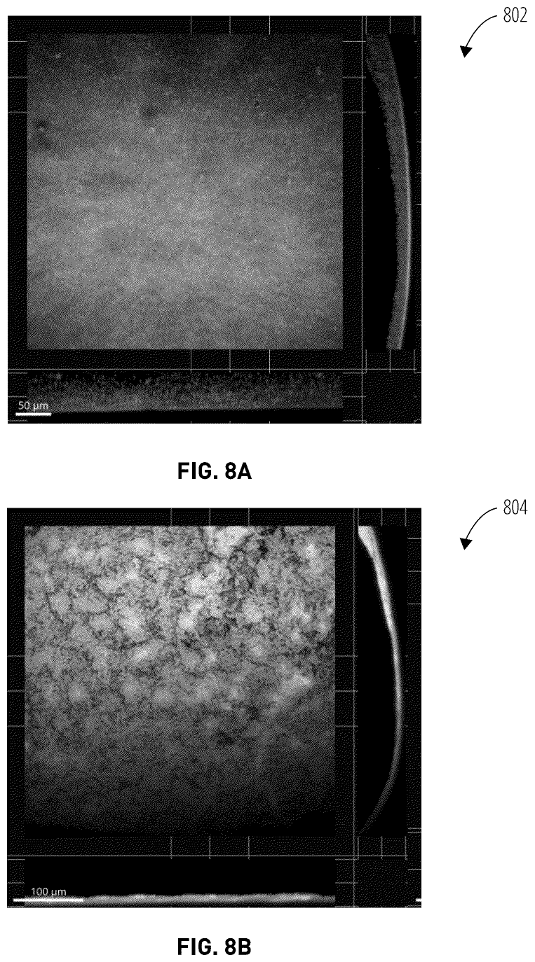

FIG. 8A shows a pre-treatment condition 802, and FIG. 8B shows a post-treatment condition 804 of a conduit 110 on which a biofilm 202 has been grown. FIG. 8A and FIG. 8B show results from treatment with an existing commercial product 412 of a conduit 110 on which a biofilm 202 was grown. While the existing commercial product 412 effectively killed the biofilm 202, the existing commercial product 412 caused either a deposit on, or etching of, the conduit 110 (shown best in FIG. 8B). The texture observed is different from that of a new, untreated conduit 110 or tube treated with the embodiment 406, embodiment 408, or embodiment 410. The texture imparted by existing commercial product 412 treatment may impact subsequent biofilm 202 regrowth by providing a niche for mechanical bacterial adhesion and subsequent colonization. Thus, the biofilm shocks 306 disclosed herein also provide the surprising benefit of killing and removing biofilms 202, but without underlying damage to the surface being cleaned, as with existing commercial products 412.

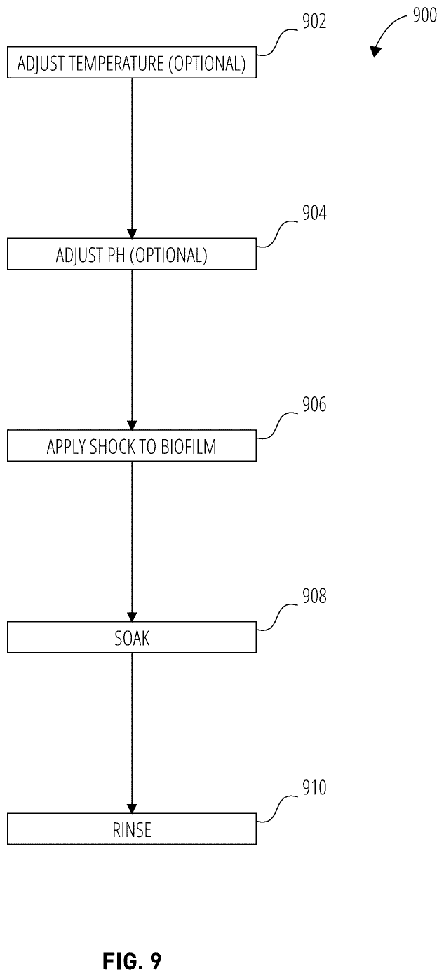

FIG. 9 illustrates an example method 900 for removing a biofilm. Although the example method 900 depicts a particular sequence of operations, the sequence may be altered without departing from the scope of the present disclosure. For example, some of the operations depicted may be performed in parallel or in a different sequence that does not materially affect the function of the method 900. In other examples, different components of an example device or system that implements the method 900 may perform functions at substantially the same time or in a specific sequence. The method 900 may make use of any embodiment of a biofilm shock disclosed herein.

According to some examples, the method 900 optionally includes adjusting temperature of the biofilm shock 306 at operation 902. In many embodiments of the method 900, the biofilm shock 306 may be adapted to be used at normal room temperature. For example, the biofilm shock 306 may be adapted to be used in a range be between about 15° C. to about 25° C. (68° F. to 77° F.). In such embodiments, the operation 902 may be optional. However, in some embodiments, the temperature of the biofilm shock 306 may be adjusted to be at higher than 25° C. in the operation 902. Such increase in temperature may help improve removing of stubborn biofilms 202 and/or reduce or soak time.

According to some examples, the method 900 includes adjusting pH at operation 904. In many embodiments, a biofilm shock 306 disclosed herein may have a pH adjusted to be in the neutral to alkaline range. For example, in embodiments that use an ammonium disinfecting agent 316 the pH may be adjusted to be about 7 to 12. In some embodiments, the pH of the biofilm shock 306 may not need adjustment (e.g., may already be in the proper range) and the operation 904 may be optional.

According to some examples, the method 900 includes applying the biofilm shock 306 to a biofilm 202 at operation 906. For example, the biofilm shock 306 may be flowed, pumped, sprayed, flushed, soaked or otherwise applied to a surface from which the biofilm 202 is to be removed. In the example of a dental water supply, the water in the supply may be drained and the biofilm shock 306 solution added. The biofilm shock 306 may be pumped through the water system via a pump, applied air pressure, or other suitable methods. For large surfaces, such as the insides of process vessels, the vessels may be filled with the biofilm shock 306 or it may be sprayed on a biofilm containing surface (e.g., the inside of the vessel).

According to some examples, the method 900 includes soaking the biofilm 202 with the biofilm shock 306 at operation 908. For example, the biofilm 202 may be wetted with the biofilm shock 306 for a certain time. In some embodiments, to keep the surface wetted, the biofilm shock 306 may be continually sprayed on the surface in the operation 908 while the surface in the is being cleaned. In some embodiments, the biofilm 202 may be wetted with the biofilm shock 306 for about 5, 10, 15, 20, 25, or 30 minutes, 1, 2, 3, 4, 5, 6, 7, or 8 hours, overnight, or up to 24 hours. The soak time may be determined based on the type of microorganism forming, or resident in, the biofilm 202, the thickness, sturdiness, etc., of the biofilm 202, the strength of the biofilm shock 306 formulation being used, the type of equipment being cleaned, the pH and/or temperature of the biofilm shock 306.

According to some examples, the method 900 includes rinsing at operation 910. For example, once a desired level of removal of the biofilm 202 has been achieved, the biofilm shock 306 may be rinsed away with a fluid such as water, another cleaner, fresh biofilm shock 306, or the like. The rinsing operation may remove or flush the biofilm 202 from the surface 104 after the biofilm 202 has been weakened and killed by the biofilm shock 306.

Any description of a particular component being part of a particular embodiment, is meant as illustrative only and should not be interpreted as being required to be used with a particular embodiment or requiring other elements as shown in the depicted embodiment.

All relative and directional references (including top, bottom, side, front, rear, and so forth) are given by way of example to aid the reader's understanding of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

The present disclosure teaches by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

What is claimed is:

1. A biofilm shock treatment, comprising:
a zinc ion source;
a disinfecting agent; wherein the biofilm shock treatment is configured to weaken a matrix of a biofilm and to disinfect causative microbes while promoting biofilm removal.

2. The biofilm shock treatment of claim 1, further comprising a chelating agent.

3. The biofilm shock treatment of claim 1, further comprising a non-ionic surfactant.

4. The biofilm shock treatment of claim 1, wherein the zinc ion source is configured to weaken the matrix of the biofilm.

5. The biofilm shock treatment of claim 1, wherein the zinc ion source comprises at least one of zinc acetate, zinc acetate dihydrate, zinc lactate, or zinc oxide.

6. The biofilm shock treatment of claim 1, further comprising lactic acid.

7. The biofilm shock treatment of claim 1, wherein the zinc ion source is between about 1.4 to 5 mass percent of the biofilm shock treatment.

8. The biofilm shock treatment of claim 2, wherein the chelating agent is between about 1.3 and 14.7 mass percent of the biofilm shock treatment.

9. The biofilm shock treatment of claim 7, wherein the disinfecting agent is between about 0.15 and 2.25 mass percent of the biofilm shock treatment.

10. The biofilm shock treatment of claim 1, wherein the disinfecting agent comprises one or more ammonium chlorides.

11. The biofilm shock treatment of claim 3, wherein the non-ionic surfactant comprises at least one of polyethylene glycol, octoxynol-9, octoxynol-10, or octoxynol-12, alkyl glucosides, alcohol ethoxylates, block polymers, or a pluronic nonionic surfactant.

12. A method of removing a biofilm, comprising:
applying a biofilm shock solution to a surface having a biofilm, the biofilm shock solution comprising:
a zinc ion source,
a disinfecting agent;
a chelating agent; and
a surfactant.

13. The method of removing a biofilm of claim 12, wherein:
the zinc ion source is between about 1.4 to 5 mass percent of the biofilm shock solution;
the chelating agent is between about 1.3 and 14.7 mass percent of the biofilm shock solution; and
the disinfecting agent is between about 0.15 and 2.25 mass percent of the biofilm shock solution.

14. The method of removing a biofilm of claim 12, wherein the surface comprises a conduit in a dental waterline system.

15. The method of removing a biofilm of claim 12, further comprising allowing the biofilm shock solution to interact with the biofilm for a predetermined period to weaken a matrix of the biofilm; and
removing the weakened biofilm from the surface.

16. A method of making a biofilm shock solution, comprising:
combining a zinc ion source;
a disinfecting agent; and
water to form the biofilm shock solution.

17. The method of making the biofilm shock solution of claim 16, further comprising combining a chelating agent into the biofilm shock.

18. The method of making the biofilm shock solution of claim 16, further comprising combining a surfactant into the biofilm shock.

19. The method of making the biofilm shock solution of claim 16, further comprising selecting the disinfecting agent based on a type of targeted microbe.

20. The method of making the biofilm shock solution of claim 16, wherein:
the zinc ion source is between about 1.4 to 5 mass percent of the biofilm shock solution;
the chelating agent is between about 1.3 and 14.7 mass percent of the biofilm shock solution; and
the disinfecting agent is between about 0.15 and 2.25 mass percent of the biofilm shock solution.
